(19) United States
(12) Patent Application Publication
(10) Pub. No.: US 2007/0003564 A1
(43) Pub. Date: Jan. 4, 2007

(54) Diluent for immunohistochemistry

(76) Inventors: John K. Hurley, Tucson, AZ (US); Anthony L. Hartman, Tucson, AZ (US)

Correspondence Address: VENTANA MEDICAL SYSTEMS, INC.;ATTENTION: LEGAL DEPARTMENT, 1910 INNOVATION PARK DRIVE, TUCSON, AZ, 85755, US

(21) Appl. No.: 11/473,941

(22) Filed: Jun. 23, 2006

Related U.S. Application Data
(60) Provisional application No. 60/696,026, filed on Jun. 30, 2005.

(51) Int. Cl. A61K 39/00; A61K 38/17; A61K 31/7012; A61K 31/045

(52) U.S. Cl. 424/184.1; 514/2; 514/53; 514/738

(57) ABSTRACT

An embodiment of the invention is directed to a method and diluent for stabilizing biomolecules, comprising Tris and a polyhydroxy carbohydrate selected from the group consisting of mannitol and trehalose. The diluent may optionally include casein added as a background blocking protein. The diluent may optionally include sodium azide as an anti-microbial agent. Mannitol and/or trehalose are particularly useful embodiments of polyhydroxy carbohydrates.

### CROSS-REFERENCE TO RELATED CASES

This application claims priority to U.S. Provisional patent application Ser. No. 60/696,026 filed 06/30/2005, the entire contents of which are incorporated by reference herein.

### BACKGROUND

1. Field of the Invention

The invention relates generally to the field of life sciences, more particularly the field of preservation and storage of proteins, and further to the field of stabilizing biomolecules for subsequent shipping and storage for later use in Immunohistochemistry (“IHC”).

2. Description of Related Art

Buffered protein solutions have long been used as diluents, or stabilizing buffered solutions, for storage and dilution of active biomolecules such as for example antibodies. Diluents also serve the function of reducing non-specific binding to containers they are in, and to tissue components they may later contact in normal analytical use. Sugars such as Sorbitol (Andersson, Maria, Protein Stabilization. Some Methods and Mechanisms, Dissertation abstract, Lund University, 1999, http://theses.lub.lu.se/postgrad/) have been used historically to improve the stability of proteins during and after the lyophilization process (essentially freeze-drying) to improve their shelf-life. Lyophilized antibodies generally have much longer shelf-lives, and in some cases may be stored indefinitely if kept in a low-temperature environment. However, antibodies stored in diluents still do not match the shelf life of a lyophilized antibody, so improved diluents are still needed.

Numerous publications in the art disclose means of increasing the stability of a lyophilized reagent, and the difficulties inherent even within this state-of-the-art technology. For example, U.S. Pat. Nos. 5,955,448, 4,496,537, and PCT filing WO 97/04801 all disclose improvements in lyophilization techniques. Lyophilization of protein-containing solutions, however, imposes major costs and inconvenience on the manufacturer and the end user, as well as introducing an increased risk of reconstitution errors and contamination. Additionally, freeze-drying of a solution requires special equipment and ultimately can lead to protein degradation when repeated cycling occurs. For commercial use, deterioration of polypeptides and antigens in aqueous solutions is costly because such solutions require replacement after only a short storage life.

PCT pub. No. WO99/15901 discloses a diluent for the stabilization of antigens, in particular, Hepatitis C Virus (HCV) antigens. The HCV diluent comprises a reducing agent to keep the HCV antigens in a reduced form. The publication reports that the inclusion of a reducing agent in a diluent maintained the immunoreactivity of an HCV antigen for up to seven days. The reported diluent further comprises sodium phosphate, pH 6.5 (or other buffer), EDTA (or other chelator), DTT (or other reducing agent), gelatin (or other protein blocking source), ammonium thiocyanate (or other chaotrope), sodium azide (or other preservative) and SDS (or other detergent).

U.S. Pat. No. 4,956,274 concerns techniques for stabilizing peptide fragments from beta-galactosidase for use in complementation assays. The solution disclosed in U.S. Pat. No. 4,956,274 contained an ionic surfactant or a surfactant derived from a sugar residue to slow degradation of beta-galactosidase peptide fragments. However, the surfactants also denatured the enzyme fragments, and thus had to be removed or neutralized to enable the enzymatic fragments to return to their correct conformation and regain enzymatic activity, indicating that the solution did not stabilize the native form of the protein. The surfactants are neutralized just prior to the assay by using cyclodextrin. Alternatively, the action of the surfactants was masked with a high concentration of serum. Additional components of the disclosed reagent included a chelating agent, buffer, bacteriocide, magnesium or other ions, reducing agents, solubilizing agents such as solvents like ethylene glycol, and nonionic detergents. As those in the art will appreciate denaturing and renaturing proteins or enzyme fragments may damage some antigenic epitopes and render them inactive.

Landi, S. and Held, H. R. (Tubercle 59 (1978) 121-133) reported the addition of TWEEN™ 20 (Polyoxyethylene Sorbitan Monooleate), detergent into a diluted solution and suggested that tuberculin PPD stability was enhanced by this addition due to the detergent's anti-adsorptive properties. The tuberculin preparation, made by Connaught Laboratories, LTD., contains tuberculin PPD, 0.3% phenol (reported to act as a preservative), and 0.0005% TWEEN™ 80 (Polyoxyethylene Sorbitan Monooleate) in PBS.

U.S. Pat. No. 6,579,688 (Steaffen et al.) discloses a diluent useful for stabilizing antigens useful in analytical procedures comprising 50 mM sodium phosphate, 2% v/v fetal calf serum, 1% v/v glycerol, 50 mM sodium chloride, 5 mM EDTA, 0.05% v/v TWEEN 20 (Polyoxyethylene Sorbitan Monolaurate) detergent, 0.01% w/v quaternary ammonium compound, and 0.5% w/v gentamycin sulfate.

### SUMMARY OF THE INVENTION

An embodiment of the invention is directed to a diluent for stabilizing biomolecules, comprising Tris and a polyhydroxy carbohydrate selected from the group consisting of mannitol and trehalose. The diluent may optionally include casein added as a background blocking protein. The diluent may optionally include sodium azide as an anti-microbial agent. Mannitol and/or trehalose are particularly useful embodiments of polyhydroxy carbohydrates. Another embodiment of the invention is a method for stabilizing biomolecules comprising storing a biomolecule in a diluent comprising Tris and a polyhydroxy carbohydrate selected from the group consisting of mannitol and trehalose.

### BRIEF DESCRIPTION OF THE DRAWINGS

FIG. 1 shows four microphotographs of tonsil sections stained with anti-Ki67 antibody using QDot 605-Streptavidin conjugate on Day 0 and Day 1 following one day of storage at 37 degrees C. MDS diluent is used on the left frames, while casein+0.08% NaN3 is used in the two frames on the right.

FIG. 2 shows four photomicrographs of anti-CD45 LCA staining of tonsil sections detected using a Qdot 605-Streptavidin conjugate. Comparison of Casein (15 mg/ml, 0.08% NaN3) with Tris buffered manitol containing bovine serum albumin, bovine gamma globulin or casein.

FIG. 3 shows four photomicrographs of anti-CD45 LCA staining of tonsil sections detected using a Qdot 605-streptavidin conjugate. Comparison of Casein (15 mg/ml, 0.08% NaN3) with Tris buffered manitol containing bovine serum albumin, bovine gamma globulin or casein.

FIG. 4 shows six Tonsil sections stained with anti-CD45 LCA diluted in either MDS antibody diluent or 50 mM Tris, 100 mM Manitol, 2 mg/ml Casein and 0.08% NaN3 and stored at 45° C.

FIG. 5 shows six photomicrographs of carcinoma sections stained with anti-pERK antibody diluted in either TBST antibody diluent or 50 mM Tris, 100 mM Mannitol, 2 mg/ml Casein and 0.08% NaN3 and stored at 45° C.

### DETAILED DESCRIPTION OF THE PREFERRED EMBODIMENTS

The diluent of the present invention comprises a Tris buffer, and a polyhydroxy carbohydrate selected from the group consisting of Mannitol and trehalose. Casein may be useful as a background blocker for subsequent staining runs. Optionally, a preservative anti-microbial such as sodium azide may be used. The preferred diluent is Tris (pH 7.2), mannitol, hydrolyzed casein and sodium azide. The diluent may be used to dilute antibodies or other biomolecules for shipment and/or storage.

Trizma® base (Tris(hydroxymethyl)aminomethane) is the preferred buffer for this diluent. It is available from many suppliers but one is Sigma-Aldrich (St. Louis, Mo.) P/N T6066. Although it may be useful in a range of concentrations, it is most preferred at 0.05 molar (50 mmol), and physiological pH (pH 6.8-7.8). pH 7.2 is preferred. pH is adjusted simply by adding concentrated HCl until the desired pH is attained.

A polyhydroxy carbohydrate is an organic compound consisting of a chain of carbon atoms to which hydrogen and oxygen are attached in a 2:1 ratio, eg, sugars, starch, glycogen, cellulose, and that contain multiple hydroxy (—OH) groups.

D-Mannitol is a sugar alcohol, available from Sigma-Aldrich, P/N M9647. The concentration range is from about 100 to about 400 mmol, and most preferred is 100 mmol. Trehalose is a naturally occurring disacharride composed of two D-glucopyranose units. It was recently reported by Lins et al, Lins, Roberto D., PROTEINS: Structure, Function and Bioinformatics 55:177-186 (2004), as a protein stabilizer. It is also available from Sigma-Aldrich, P/N T9531 (dihydrate form).

An anti-microbial agent is optionally included. One which has been found to not interfere with IHC requirements is sodium azide. It is available from many suppliers, including Sigma, P/N S-2002. The least amount appropriate to retard microbial growth was used since it is generally toxic, and can be explosive under certain circumstances. 0.05 wt./vol. % 0.1% is a generally preferred range, and 0.08% is a preferred concentration used herein.

A background blocking agent is generally used with antibody diluents so that non-specific protein-protein binding is minimized in subsequent staining runs. Non-specific protein-protein binding may occur between the primary antibody and many other proteins in the tissue sample, so some method of quenching this “stickiness” should be used to inhibit background signal. Many proteins can be used for this, including Bovine Serum Albumin (BSA), Bovine Gamma Globulin (BGG) and hydrolyzed casein. Particularly preferred is hydrolyzed casein from American Casein Co. (Burlington, N.J.), P/N HCA-411. Concentrations of from 2 mg/ml to 15 mg/ml are useful, although 2 mg/ml is preferred. One of ordinary skill in the histology art will know of many interchangeable blocking proteins that can be utilized. Individual blocking buffers are not compatible with every system. For this reason, a variety of blockers in both Tris buffered saline (TBS) and phosphate buffered saline (PBS) are available. The proper choice of blocker for a given tissue depends on the antigen itself and on the type of enzyme conjugate to be used. For example, with applications using an alkaline phosphatase conjugate, a blocking buffer in TBS should be selected, because PBS interferes with alkaline phosphatase. The ideal blocking buffer will bind to all potential sites of nonspecific interaction, eliminating background altogether without altering or obscuring the epitope for antibody binding. For true optimization of the blocking step for a particular immunohistochemical assay, empirical testing is essential. Many factors, including various protein:protein interactions unique to a given set of immunoassay reagents, can influence nonspecific binding. The most important parameter when selecting a blocker is the signal:noise ratio, measured as the signal obtained with a tissue sample containing the target analyte, as compared to that obtained with a sample without the target analyte. Using inadequate amounts of blocker will result in excessive background staining and a reduced signal:noise ratio. Using excessive concentrations of blocker may mask antibody:antigen interactions or inhibit the marker enzyme, again causing a reduction of the signal:noise ratio. When developing any new immunoassay, it is important to test several different blockers for the highest signal:noise ratio in the assay. No single blocking agent is ideal for every occasion since each antibody-antigen pair has unique characteristics. Agents such as nonfat dry milk and salmon serum, in addition to those mentioned above, may be used.

Formulation 1 is 50 mmol Trizmag HCl, 100 mmol mannitol, 2 mg/ml hydrolyzed casein and sodium azide 0.08%. This formulation was used in the examples below to dilute the antibodies mentioned. It is referred to as the “TBMC” diluent. Prior art diluent MDS is 77 mM Sodium Phosphate, dibasic; 23 mM Sodium Phosphate, monobasic; 54.75 mM Sodium Chloride; 0.05% Brij 35; 15.0 mg/ml Hydrolyzed Casein Protein; 0.05% ProClin 300. TBST diluent is 19.98 mM Trizma® Base; 136.89 mM Sodium Chloride; 0.1% Tween 20; 5% Normal Goat Serum (Chemicon International, Temecula, Calif., P/N S26).

### EXAMPLES

### Example 1

### LCA, pERK ½ Antibody Stability

The stabilities of two antibodies, anti-pERK ½ and LCA, were compared to their stability in the novel TBMC diluent, and the prior art MDS and TBST diluents, at the elevated temperature of 45° C., a standard ship-stress test temperature. Anti-CD45 Leucocyte Common Antigen (LCA) clone 2B11 from Neomarkers, Inc., Fremont, Calif., P/N MS-1884-P0 was used. 4 μm tonsil sections were prepared for staining on a DISCOVERY® automated tissue stainer (Ventana).

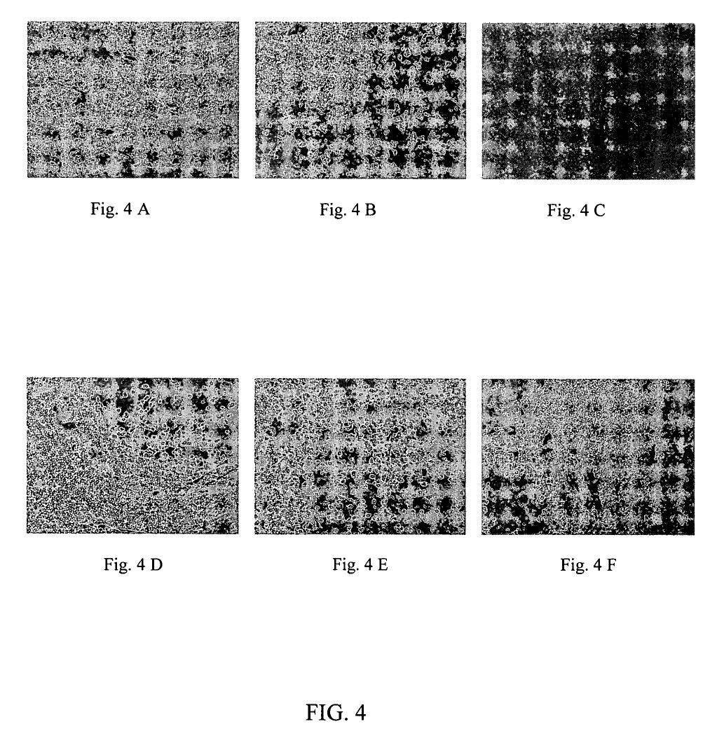

The present MDS diluent is hostile to the stability of phosphorylated antibodies, as seen in FIG. 4 in the top three frames labeled “MDS Diluent Day X”, where X is 0 in the first frame, and increases with increasing time in the diluent to 14 days in the last frame. By the last frame (Day 14) all antibody activity is gone in the MDS diluent, whereas in the TBMC diluent the LCA antibody is still staining the tonsil sections well.

We then looked at the stability of phospho-ERK½ (“pERK”) from Biosource International, Camarillo, Calif., P/N 44-680G in the TBMC diluent and in TBST diluent.

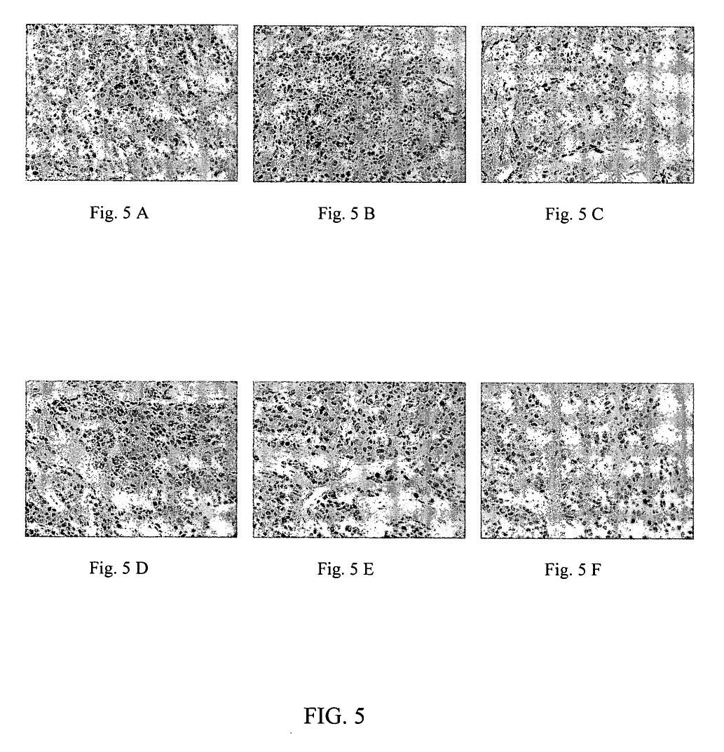

In FIG. 5, the top three frames show the pERK antibody diluted in TBMC diluent, and in TBST diluent (bottom three frames) at time=0, 7 days, and 14 days. Stability in both diluents is apparent. The sections stained were 4 um breast carcinoma sections sourced from the in-house Ventana tissue bank. However, those of ordinary skill will appreciate that any breast carcinoma tissue expresses pERK, and so any breast tumor tissue may be used.

### Example 2

### Stability of Quantum Dot Conjugates

“Quantum Dots” are semiconductor nanocrystal-based fluorescent tags that are being used in life sciences research due to their ability to emit narrow frequencies of light when appropriately excited. They can be conjugated to biomolecules such as haptens, antibodies or DNA probes, and then bound or hybridized to their respective biological targets.

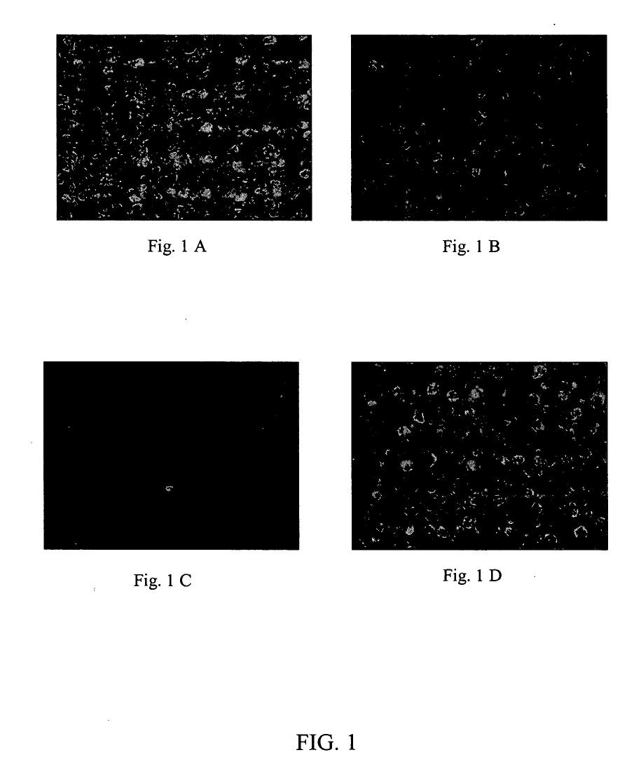

FIG. 1 shows the problems encountered with the prior art MDS diluent with the QDot®-Streptavidin conjugate when used to detect the primary antibody anti-Ki-67, clone K-2 (Ventana P/N 790-2910). 4 um tonsil sections were prepared for staining on a DISCOVERY system, as before. QDot 605 conjugated to Streptavidin (Invitrogen Corp., Carlsbad, Calif., P/N Q21301MP) was diluted into both the prior art MDS diluent, and a casein/Sodium azide diluent (2 ug/ml hydrolyzed casein; 0.08% NaN3). The two left frames show tonsil sections stained with the Ki67/QDot 605 combination diluted into the MDS diluent on Day 0. (top-left) and Day 1 (bottom left). The signal clearly declines rapidly to being non-existent on Day 1. By contrast, switching to a casein-only diluent reduces the fading of the QDot 605 signal greatly so that there is no apparent fading between Day 0 and Day 1. Clearly, the MDS diluent is not friendly to QDot conjugate stability at 37° C.

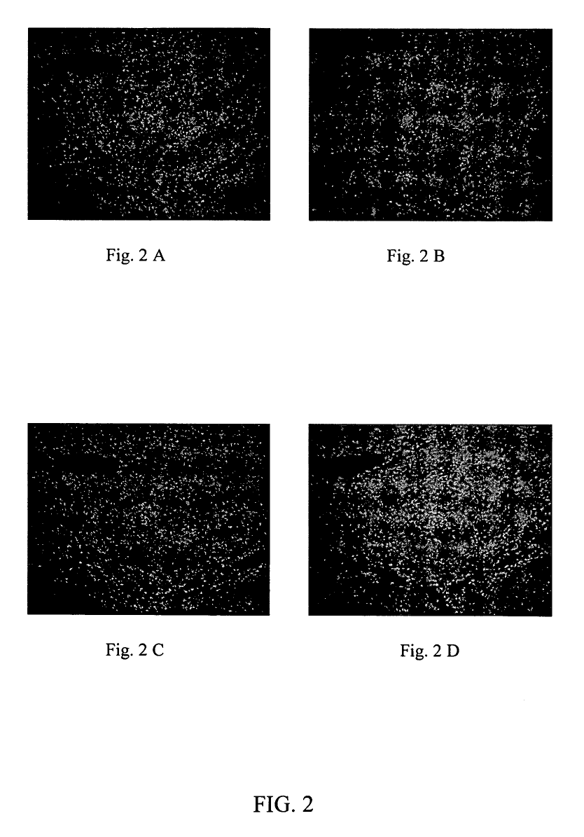

FIG. 2 shows the effect casein seems to have in the TBMC diluent. The figure shows a different antibody anti-CD45 antibody LCA being stained on tonsil sections, and then being detected using the same QDot-Streptavidin 605 conjugate. This figure shows that the blocking proteins behave surprisingly differently regarding stability of the diluent. For instance, the signals degrade most rapidly in BGG (bovine gamma globulin, bottom right), then degrade significantly in BSA (bovine serum albumin, top right) then not at all in casein (top left). These results were generated at 45° C.

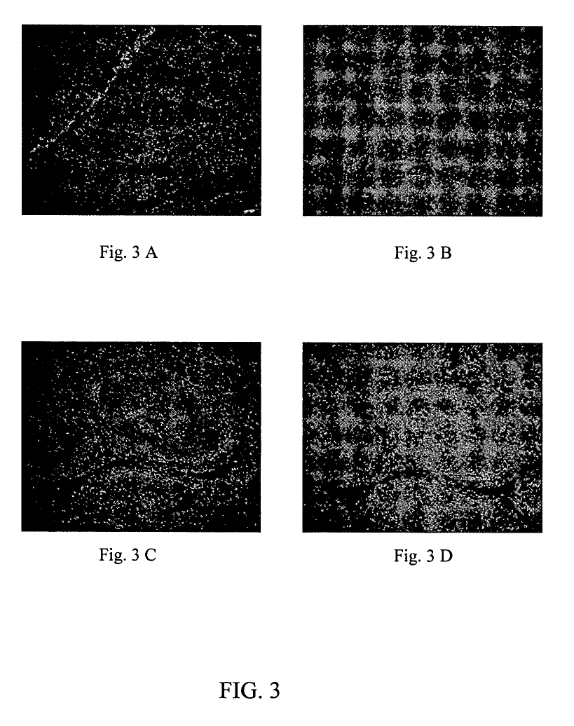

FIG. 3 is a longer term (5 days versus two) and higher temperature (45 v. 37) test of the TBMC diluent discussed in FIG. 2. The results are similar, with increased degradation of signal over the extra time and higher temperatures in all diluents.

While the invention has been described with reference to an illustrative embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiments disclosed as the best mode contemplated for carrying out this invention, but that the invention will include all embodiments falling within the scope of the appended claims.

We claim:

1. A diluent for stabilizing biomolecules, comprising Tris and a polyhydroxy carbohydrate selected from the group consisting of mannitol and trehalose.

2. The diluent of claim 1 wherein casein is optionally added as a background blocking protein.

3. The diluent of claim 1 wherein the Tris concentration is effective to buffer the pH to between about 6.8 and about 7.8.

4. The diluent of claim 1 wherein the concentration of the polyhydroxy carbohydrate ranges from about 100 to about 400 mmol.

5. The diluent of claim 2 wherein the concentration of casein ranges from about 2 mg/ml to about 15 mg/ml.

6. The diluent of claim 1 wherein optionally is included sodium azide, and the concentration of sodium azide ranges from about 0.05 to about 0.10%, wt./vol.

7. The diluent of claim 1 wherein the Tris concentration is about 50 mM.

8. The diluent of claim 1 wherein the mannitol concentration is about 100 mM.

9. The diluent of claim 2 wherein the casein is about 2 mg/mi.

10. The diluent of claim 6 wherein the sodium azide is about 0.08% wt./vol.

11. A diluent for stabilizing biomolecules in aqueous solution, comprising 50 mmol Tris, 100 mmol mannitol, 2 mg/ml casein and sodium azide 0.08%.

12. A method for stabilizing a biomolecule comprising storing a biomolecule in the diluent of claim 1.
